(19) United States
(12) Patent Application Publication
(10) Pub. No.: US 2019/0307438 A1
(43) Pub. Date: Oct. 10, 2019

(54) CATHETER ATRIAL SEPTAL CLOSURE DEVICE

(71) Applicant: TAVR Solutions, LLC, St. Louis, MO (US)

(72) Inventors: Alan Zajarias, Olivette, MO (US); Sara Jane Gries, Osseo, MN (US)

(21) Appl. No.: 16/380,488

(22) Filed: Apr. 10, 2019

Related U.S. Application Data
(60) Provisional application No. 62/655,318, filed on Apr. 10, 2018.
(60) Provisional application No. 62/674,343, filed on May 21, 2018.

(51) Int. Cl. A61B 17/00

(52) U.S. Cl. CPC A61B 17/0057; A61B 2017/00022; A61B 2017/00526; A61B 2017/00623; A61B 2017/00606; A61B 2017/00867; A61B 2017/00597; A61B 2017/00592

(57) ABSTRACT

The present invention is directed to an apparatus, system, and method for occluding or closing bodily passageways, including septal apertures in the heart, that employs a self-expanding shape-memory material to engage and conform to the lateral faces of the bodily passageways, wherein a structure comprising the self-expanding shape-memory material extends substantially around the periphery of a membrane or a membranous covering and is designed to hold the membrane or the membranous covering generally taut. The structure comprising a plurality of notches or cut-outs and assuming a variety of shapes and sizes suitable for occluding or closing various bodily passageways, including septal apertures located low in a heart or the septum. The present invention being specifically adapted for having a reduced propensity for tearing and rupturing, permitting post-deployment transseptal punctures and interatrial re-entry, and being designed to not interact or interfere with the tricuspid or mitral valves of the heart.

### CROSS-REFERENCE TO RELATED APPLICATIONS

This application claims priority to U.S. Provisional Patent Application Ser. No. 62/655,318, filed on Apr. 10, 2018, to Alan Zajarias, entitled “Catheter Atrial Septal Closure Device,” currently pending, and U.S. Provisional Patent Application Ser. No. 62/674,343, filed on May 21, 2018, to Alan Zajarias and Sara Jane Gries, entitled “Catheter Atrial Septal Closure Device,” currently pending. The entire disclosures, including the specifications and drawings, of the above-referenced applications are incorporated herein by reference.

### FIELD OF THE INVENTION

The present invention relates generally to a medical device and method and, more particularly, to a device and method for occluding or closing a bodily passageway or aperture in body tissue, including congenital heart defects. The present invention is specifically directed to a collapsible and deployable atrial septal occlusion or closure device that may be delivered through a delivery device, including a catheter or sheath, and the associated method for deploying the same.

### BACKGROUND OF THE INVENTION

The heart has two sides separated by two inner dividing walls, or septa, which are known as the interatrial septum and the interventricular septum. The right side of the heart receives oxygen-poor blood from the body and pumps it into the lungs, where it is oxygenated. The left side of the heart receives the oxygen-rich blood from the lungs and pumps it to the body. The interatrial septum separates the upper chambers of the heart, and the interventricular septum separates the bottom chambers of the heart. Each septum serves to prevent the mixing of blood between the right and left sides of the heart.

Apertures, or holes, in the septa of the heart are defects that can affect the normal flow of blood through the heart. Such apertures can occur congenitally or be caused by puncturing, including puncturing by a medical device or the like. An aperture in the interatrial septum between the heart's two upper chambers is known as an atrial septal defect (“ASD”), with secundum ASDs being the most common form of ASDs. An ASD can cause the mixing of oxygen-rich blood with oxygen-poor blood. Such mixing of blood with differing oxygen contents can cause blood with high oxygen content to be pumped to the lungs rather than the body, and blood with low oxygen content to be pumped to the body rather than the lungs, which is known as blood shunting. Depending upon the size of the aperture and the amount of blood shunting, this can result in a spectrum of diseases including, without limitation, abnormal heart rhythms, abnormal elevation in blood pressure in the pulmonary arteries, and congestive heart failure.

Surgical intervention techniques to treat ASDs, including open-heart surgery techniques, have evolved over the past several decades. Initial attempts to surgically occlude or close ASDs employed various failed techniques and devices, such as the use of stiff polythene buttons to invaginate atrial appendages via transatrial sutures and to occlude or close the ASD. Further, open-heart surgery typically requires restricting or bypassing blood flow through the heart during surgery. Recently, use of a heart-lung machine, or cardiopulmonary bypass (“CPB”), has become the gold standard of bypassing the heart temporarily for open-heart surgery techniques, including ASD repair. Nonetheless, open-heart surgery techniques that use the CPB, including for ASD repair, present a number of disadvantages, including patient pain, prolonged recovery time, invasive procedures, significant scarring on the patient's body, and other drawbacks. Therefore, surgical intervention techniques to treat ASDs have been ineffective and overly invasive, such that a need existed for treatment techniques for ASDs that were more effective and less invasive.

Over the past few decades, less invasive treatment techniques have been developed, and transcatheter treatment techniques have become preferred techniques for treating ASDs and avoiding the adverse side effects common to other treatment techniques. Specifically, percutaneous transcatheter treatment techniques provide for a safer and less invasive medical procedure. However, occlusion or closure devices used in known percutaneous transcatheter treatment techniques are not without their disadvantages. A number transcatheter occlusion or closure devices generally employ umbrella-like structures to occlude or close ASDs. Known occlusion or closure devices have a number of specific disadvantages, including, but not limited to, a propensity to tear or fracture, a propensity to perforate body tissue, including heart tissue, a propensity for residual leaking, an elevated risk of complications due to thrombus, a tendency to erode the atrial and aortic walls, and other drawbacks. Further, many known occlusion or closure devices have high profiles and include large masses of foreign material, such as an excess amount of occlusion or closure membrane or fabric, that may impair the adaptation of the device by the patient's body.

Another disadvantage of known occlusion or closure devices is that, in cases where the patient may need a subsequent transseptal procedure, the presence of the deployed or implanted device generally inhibits the ability to transseptally puncture the device for purposes of recrossing the septum or permitting interatrial re-entry. This disadvantage results from the fact that the structure of the deployed or implanted device may block the passage of certain-sized sheathes or other medical instruments through the septum and/or comprises materials that are incapable of permitting single or repeated transseptal punctures or interatrial re-entry therethrough.

Another disadvantage of known occlusion or closure devices is their limited ability, or complete inability, to occlude or close holes or apertures that are located relatively low in a heart or its septum, because of the potential interaction or interference of such occlusion or closure devices with the tricuspid or mitral valves.

Accordingly, a need exists for a minimally invasive device and method for occluding or closing a bodily passageway or aperture, including congenital heart defects such as ASDs. Such a device and method being designed for use in surgical intervention to treat congenital heart defects and may employ a self-expanding shape-memory material that has sufficient shape-memory to maintain the device's intended shape once it is released from a delivery device or deployed. The device and method having a reduced propensity for tearing or rupturing, permitting transseptal punctures or interatrial re-entry, and being designed to not interact or interfere with the tricuspid or mitral valves of the heart. A need also exists for an occlusion or closure device and associated method that employs opposing and generally circular elements that engage with the interatrial wall at an angle, which can allow the device to repair abnormal apertures or communications in the heart that have previously been very difficult to repair because of the interaction or interference with surrounding structures such as valves, veins, the aorta, and the like. Further, a need exists for a device and method for treating heart disease and ailments, including ASDs, patent foramen ovales, and other arterio-venuous communications, that improves over existing technology.

The present invention is designed to address these and other disadvantage of known occlusion or closure devices. Further, other desirable features and characteristics of the present invention will become apparent from the subsequent detail description and the appended claims, taken in conjunction with the accompanying figures and this section.

### SUMMARY OF THE INVENTION

The occlusion or closure device and method that is the subject of the present invention makes up for the shortcomings in the prior art. Generally, the occlusion or closure device and method hereof preferably employs a self-expanding shape-memory material to engage and conform to the lateral faces of bodily passageways, wherein the shape-memory nature of the material is sufficient to maintain the occlusion or closures device's intended shape once it is released from a delivery device or deployed for sealingly engaging the bodily passageway. The occlusion or closure device further comprising a membrane or membranous covering and a plurality of notches or cut-outs.

An object of the present invention is to provide a collapsible and deployable occlusion or closure device adapted for having a reduced propensity for tearing and rupturing. The occlusion or closure device employing a membrane or membranous covering suitable for the purposes of the present invention. The occlusion or closure device further employing a plurality of notches or cut-outs that reduce the strain and related fatigue on the occlusion or closure device and the membrane or membranous covering that could result from the cyclic loading of forces on the same.

It is a further object of the present invention to provide an occlusion or closure device and method adapted for permitting post-deployment transseptal punctures and interatrial re-entry. The occlusion or closure device employing a membrane or membranous covering comprising a material with certain resiliency and durability that allow for post-deployment medical punctures without jeopardizing the integrity of the membrane or the membranous covering or the occlusion or closure device.

It is another object of the present invention to provide an occlusion or closure device and method being designed to not interact or interfere with the tricuspid or mitral valves of the heart.

It is yet another object of the present invention to provide a occlusion or closure device and method that employs opposing and generally annular elements that engage with the interatrial wall at an angle, which can allow the device to repair abnormal apertures or communications in the heart that have previously been very difficult to repair because of the interaction or interference with surrounding structures such as valves, veins, the aorta, and the like. Engaging a circular element with the interatrial wall at an angle advantageously allows for another circular element to be properly deployed and implanted.

It is another object of the present invention to provide an occlusion or closure device and method that does not require the inclusion of large amounts or masses of foreign material and has a low propensity for perforating heart tissue, causing residual leaking, causing complications associated with thrombus, and eroding atrial walls or the aorta artery.

Still a further object of the present invention is to provide an occlusion or closure device and method that is suitable for closure of atrial septal defects, patent foramen ovales, and other abnormal arterio-venuous communications, is minimally invasive, and reduces the risk of complications associated with the delivery, deployment, and/or retrieval of the occlusion or closure device.

### BRIEF DESCRIPTION OF THE DRAWINGS

For a better understanding of the various embodiments of the present invention, reference may be made to the accompanying drawings in which:

FIG. 1 is a schematic representation of a human heart including an ASD;

FIG. 2 is a top view of an occlusion or closure apparatus according to the teachings of one embodiment of the present invention;

FIG. 3 is a perspective view of the occlusion or closure apparatus of FIG. 2 further comprising a membrane or membranous covering according to the teachings of one embodiment of the present invention;

FIG. 4 is a top view of the occlusion or closure apparatus of FIG. 3;

FIG. 5 is a cross-sectional side view of the occlusion or closure apparatus of FIGS. 3 and 4 deployed and implanted adjacent to a bodily passageway, such as an ASD;

FIG. 6 is a top view of an occlusion or closure system in a first position according to the teachings of one embodiment of the present invention;

FIG. 7 is a perspective view of the occlusion or closure system of FIG. 6 in a second position;

FIG. 8 is a top view of the occlusion or closure system in the second position of FIG. 7;

FIG. 9 is a side plan view of the occlusion or closure system in the second position of FIGS. 7 and 8;

FIG. 10 is a perspective view of the occlusion or closure system in a second position of FIGS. 7-9 further comprising a membrane or membranous covering according to the teachings of one embodiment of the present invention;

FIG. 11 is a top view of the occlusion or closure system of FIG. 10;

FIG. 12 is a cross-sectional representation of the occlusion or closure system of FIGS. 10 and 11 that is contained in a delivery device, such as a catheter or sheath, according to the teachings of one embodiment of the present invention;

FIG. 13 is a cross-sectional side view of the occlusion or closure system in the second position of FIGS. 10 and 11 deployed and implanted adjacent to a bodily passageway, such as an ASD;

FIG. 14 is a top view of an occlusion or closure system in a first position according to the teachings of another embodiment of the present invention;

FIG. 15 is a perspective view of the occlusion or closure system of FIG. 14 in a second position;

FIG. 16 is a top view of the occlusion or closure system in the second position of FIG. 15;

FIG. 17 is a side plan view of the occlusion or closure system in the second position of FIGS. 15 and 16;

FIG. 18 is a side plan view of the occlusion or closure system in the second position of FIGS. 15-17;

FIG. 19 is a perspective view of the occlusion or closure system in a second position of FIGS. 15-18 further comprising a membrane or membranous covering according to the teachings of one embodiment of the present invention;

FIG. 20 is a top view of the occlusion or closure system of FIG. 19;

FIG. 21 is a cross-sectional side view of the occlusion or closure system in the second position of FIGS. 19 and 20 deployed and implanted adjacent to a bodily passageway, such as an ASD;

FIG. 22 is a top view of an occlusion or closure system in a first position according to the teachings of another embodiment of the present invention;

FIG. 23 is a perspective view of the occlusion or closure system of FIG. 22 in a second position;

FIG. 24 is a top view of the occlusion or closure system in the second position of FIG. 23;

FIG. 25 is a side plan view of the occlusion or closure system in the second position of FIGS. 23 and 24; and

FIG. 26 is a flowchart of an exemplary embodiment of a method for occluding or closing a bodily passageway, and that may be implemented using the occlusion or closure devices of FIGS. 2-25

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure.

### DETAILED DESCRIPTION OF THE INVENTION

The invention is now to be described with reference to the figures, in which like reference numerals refer to like parts throughout. For purposes of clarity in illustrating the characteristics of the present invention, proportional relationships of the elements have not necessarily been maintained in the figures. It will be understood that any dimensions included in the figures are simply provided as examples and dimensions other than those provided therein are also within the scope of the invention.

The description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The present invention is defined by the appended claims and the description is, therefore, not to be taken in a limiting sense and shall not limit the scope of equivalents to which such claims are entitled.

One objective of the present invention is to provide an apparatus, system, and method for occluding or closing bodily passageways, including septal apertures, or holes, in the heart, and treating heart disease and ailments, including ASDs, patent foramen ovales, and other arterio-venuous communications. The apparatus, system, and method may employ a self-expanding shape-memory material that has sufficient shape-memory to maintain the device's intended shape once it is released from a delivery device or deployed, where such intended shape is designed to engage and conform to the lateral faces of a bodily passageway. In one embodiment, the apparatus, system, and method may employ opposing and generally circular elements that engage with the interatrial wall of the heart at an angle. The present invention also being releasable, deployable, and retrievable, having a reduced propensity for tearing or rupturing, permitting post-deployment transseptal punctures and interatrial re-entry, and being designed to not interact or interfere with the tricuspid or mitral valves. The present invention does not require the inclusion of large amounts or masses of foreign material, including membranes or membranous coverings. The present invention also has a low propensity for perforating heart tissue, causing residual leaking, causing complications associated with thrombus, and eroding atrial walls or the aorta artery. Further, another objective of the present invention is to provide a method for deploying such an apparatus and system that is minimally invasive, reduces the risk of complications associated with the delivery, deployment, and/or retrieval of the apparatus and system, and does not require use of CPB. One of ordinary skill in the art will understand that the apparatus, system, and method of the present invention may be useful in treating other health conditions not specifically disclosed herein.

The present disclosure describes an apparatus, system, and method suitable for occluding or closing a bodily passageway, including an ASD or patent foramen ovale, that is generally designed to, among other things, (i) provide a structure comprising a continuous piece of biocompatible material, with a membrane or membranous covering and a plurality of notches or cutouts, that is formed or heatset to allow for a first, generally elongated position and a second, biased released or deployed position, (ii) allow for such a structure or frame to be selectively released, deployed, and retrieved, and (iii) permit post-deployment transseptal punctures and interatrial re-entry through a septum.

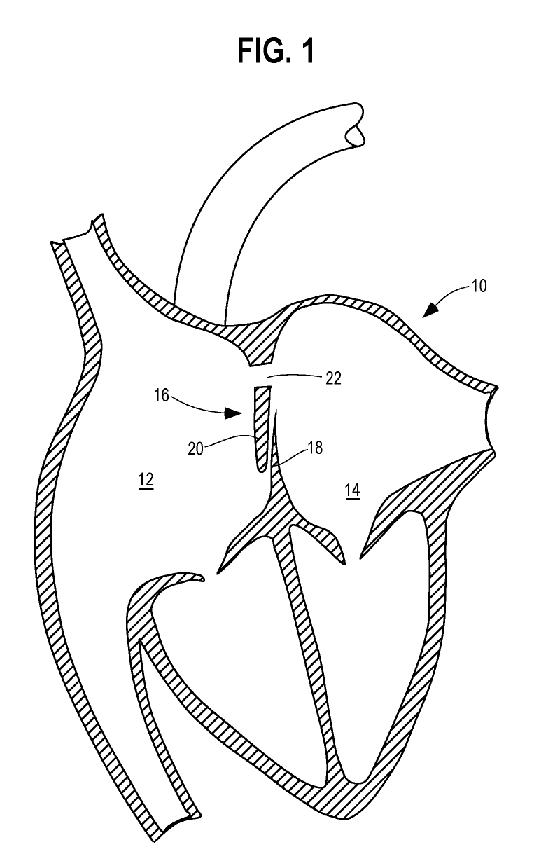

FIG. 1 illustrates a human heart 10, having a right atrium 12, a left atrium 14, an atrial septum 16, and a septal aperture 22. The atrial septum 16 generally comprising a septum primum 18 and septum secundum 20. The anatomy of the septum 16 can vary widely within a population. For example, in some people, the septum primum 18 may be quite thin. As used herein, “left” refers to the left chambers of the heart 10, including the left atrium 14 and left ventricle, and “right” refers to the right chambers of the heart 10, including the right atrium 12 and right ventricle.

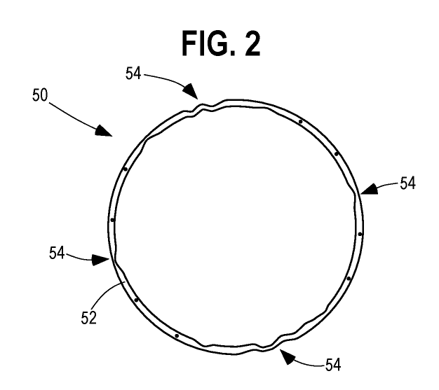

As illustrated in FIG. 2, according to one embodiment, the apparatus 50 of the present invention can comprise a continuous frame 52 formed of a wire or tubular structure. The diameter or thickness of the wire or tubular structure may be generally proportional to the size of the apparatus 50. The frame 52 can be formed of a flexible and manipulatable biocompatible or bioresorbable material or polymer, including, without limitation, iron, magnesium, platinum, stainless steel, cobalt-chromium-nickel alloy, or nickel titanium metal alloy, such as nitinol. In a preferred embodiment, the frame 52 can comprise a plurality of notches or cut-outs 54 along its length and at regular or irregular intervals. In one embodiment, the frame 52 can be collapsible or capable of being distorted so that it can be, among other things, deployable via transcatheter techniques. In this way, nitinol is preferable material for the frame 52 because of its unique characteristics of well-defined shape-memory, pronounced elasticity, and resiliency. These characteristics are desirous because they allow the apparatus 50 to be distorted in form and then to resume or return to an intended shape. For example, such characteristics allow for the apparatus 50 to be compressed during the delivery process, while returning to its intended shape in vivo. As used herein, “shape-memory” refers to a property of materials with a propensity to resume and maintain an intended shape despite being distorted for periods of time, such as during storage or during the delivery process. It will be understood that the frame 52 can comprise or be formed of any suitable material, polymer, or combination of materials and/or polymers that is, at the least, biocompatible and capable of adequate shape-memory.

The frame 52 can be any symmetrical or asymmetrical geometric shape or size suitable to achieve the purposes of the present invention. In one embodiment of the present invention, the frame 52 may generally define an annular or substantially closed shape. The frame 52 may, in some embodiments, generally define a plane. In another embodiment, the shape and size of the frame 52 can correspond with the shape and size of the subject bodily passageway, including a septal aperture 22, and may vary depending on the location of the bodily passageway in the body. For example, the shape and size of the apparatus 50 may be designed to avoid any potential interaction or interference with surrounding bodily and/or medical structures, including, without limitation, the mitral valve, tricuspid valve, and pulmonary veins. Preferably the diameter of the frame 52 can range from 10 mm to 40 mm in size. However, it will be understood that the diameter of the frame 52 can range from 50 mm to 4 mm. In one embodiment, the flexibility of the frame 52 allows for it to be expanded, contracted, and positionally adjusted to accommodate bodily passageways of varying shape and size.

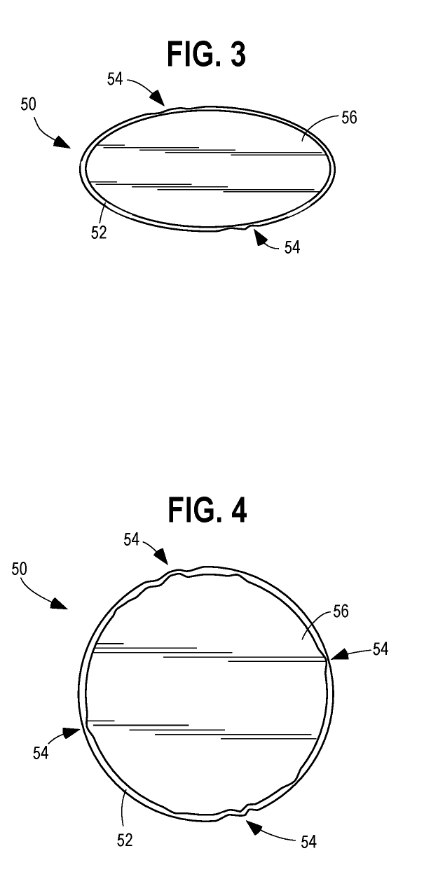

As best illustrated in FIGS. 3 and 4, in some embodiments, the apparatus 50 may further comprise a flexible biocompatible membrane or membranous covering 56 attached to and covering at least a portion of the frame 52, which is adapted to adequately occlude or close the subject bodily passageway. In one embodiment, the frame 52 is attached to and desirably extends substantially around the periphery of the membrane or the membranous covering 56 and is designed to hold the membrane or the membranous covering 56 generally taut. The membrane or membranous covering 56 may be attached to the frame 52 by any suitable attachment means presently known or later discovered, including, without limitation, via sutures, an adhesive, gluing, welding, or the like. For example, in one embodiment, the frame 52 may comprise holes spaced apart, preferably approximately 2 mm to 3 mm apart, that allow for the membrane or membranous covering 56 to be attached to the frame 52.

The membrane or membranous covering 56 may comprise expanded polytetrafluoroethylene or a bioremodelable material, such as polytetrafluoroethylene (PTFE), expanded polytetrafluoroethylene (ePTFE), polyester fabrics, or Teflon-based materials. In another embodiment, the membrane or membranous covering 56 may comprise a silk, nylon, silicone, polyethylene, polypropylene, or fluoropolymer membrane. However, the membrane or membranous covering 56 preferably comprises a biocompatible material that does not produce a significant inflammatory response or calcification response, including a PTFE, ePTFE, polyethylene terephthalate (PET), or other polyethylene membrane, pericardium, or other polymer. Such materials being advantageous because of their thickness, durability, and stretchability. In a preferred embodiment, the membrane or membranous covering 56 is adapted to inhibit the passage of blood while also permitting post-deployment transseptal punctures and interatrial re-entry. According to some embodiments, the membrane or membranous covering 56 can comprise any natural, synthetic, or bioengineered material, whether presently known or later discovered, that resiliently occludes or closes the subject bodily passageway and is also sufficiently durable to allow for post-deployment medical punctures without jeopardizing the integrity of the membrane or the membranous covering 56, the apparatus 50, or the apparatus's 50 ability to occlude.

The plurality of notches or cut-outs 54 of the frame 52 permit for certain material reaction and behaviors that generally reduce the strain and related fatigue on the frame 52 and the membrane or membranous covering 56 that could result from the cyclic loading of forces on the same. This resulting reduction of strain and fatigue on the frame 52 and the membrane or membranous covering 56 greatly reduces the propensity for the apparatus 50 to tear or fracture, which is an improvement over known occlusion or closure devices. The plurality of notches or cut-outs 54 can further facilitate the additional flexibility of the system 100. Specifically, the plurality of notches or cut-outs 54 allow the frame 52 to selectively deform more easily, which is advantageous for purposes of overcoming the limited flexibility of the membrane or membranous covering 56 or other elements of the system 100. Such flexibility can allow the system 100 to adequately and repeatedly move between various positions without requiring of the membrane or membranous covering 56 to be slack or comprise excess or additional material for purposes of overcoming its limited flexibility.

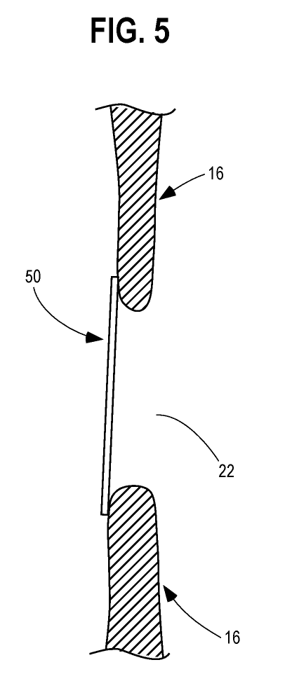

As best illustrated in FIG. 5, according to one embodiment of the present invention, to occlude or close a bodily passageway, including a septal aperture 22 in the heart 10, the apparatus 50 can be adapted to sealingly engage and/or conform to the lateral faces of the bodily passageway through a variety of means, including, without limitation, transatrial sutures or other suitable attachment means presently known or later discovered. Further, in another embodiment, the apparatus 50 may contain one or more sensors, including, but not limited to electrical sensors, pressure sensors, biochemical sensors, impact sensors, strain gauges, accelerometers, non-physical contact sensors, acoustic sensors, infrared sensors, and other sensor.

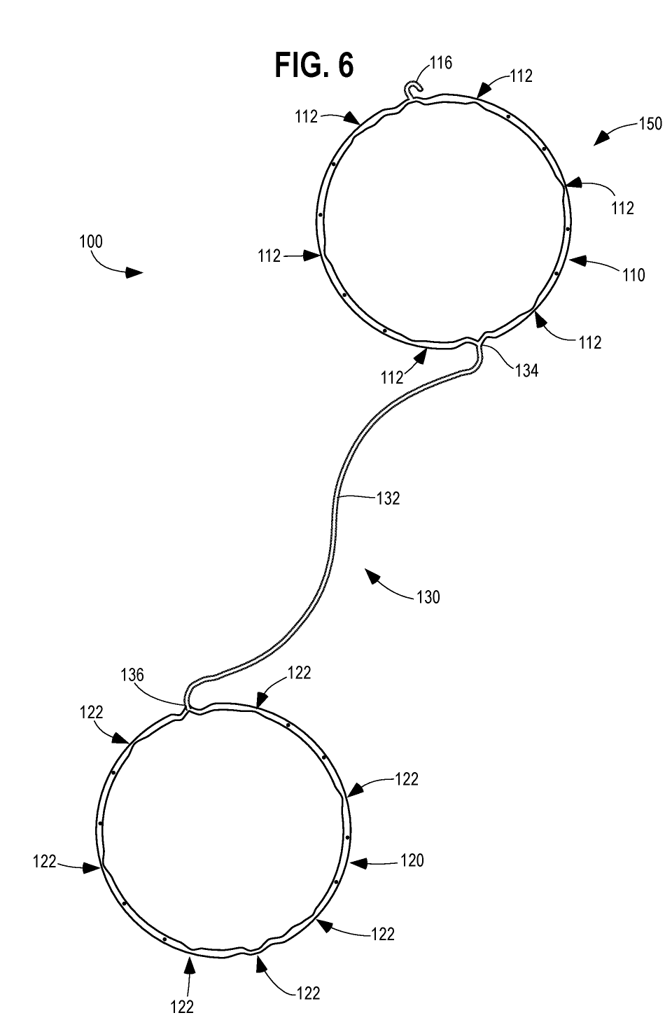

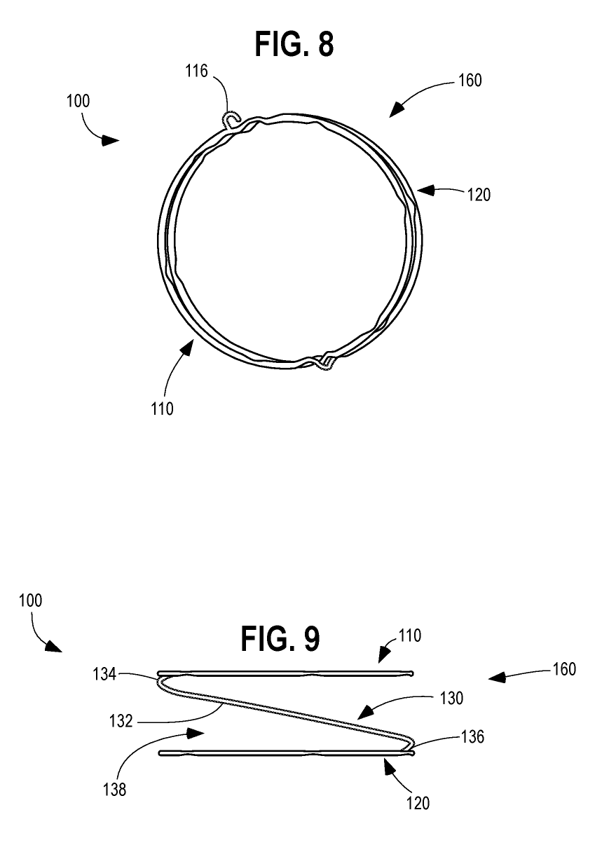

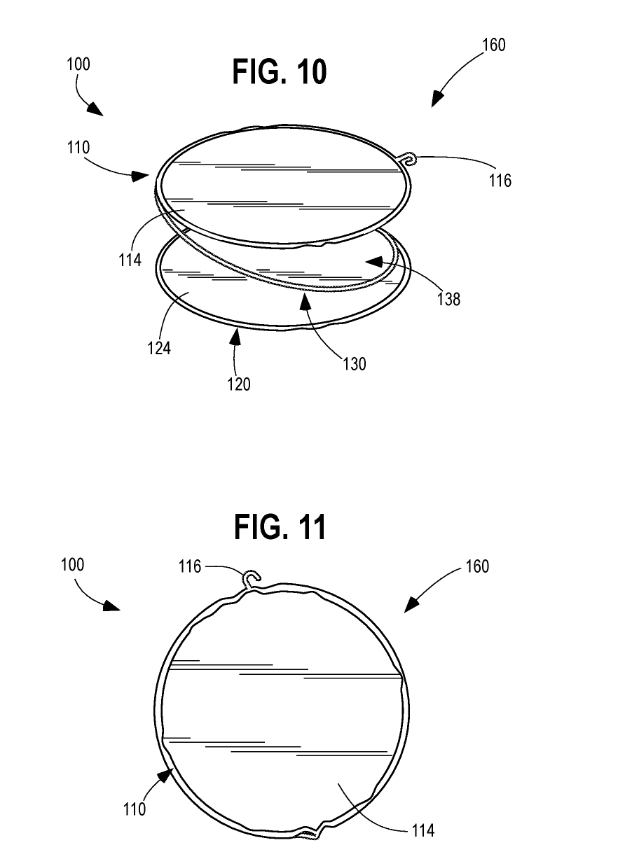

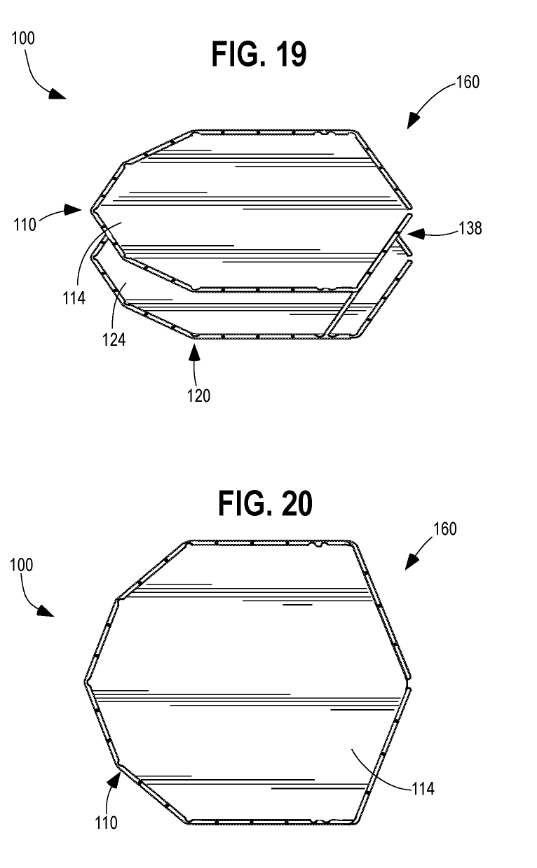

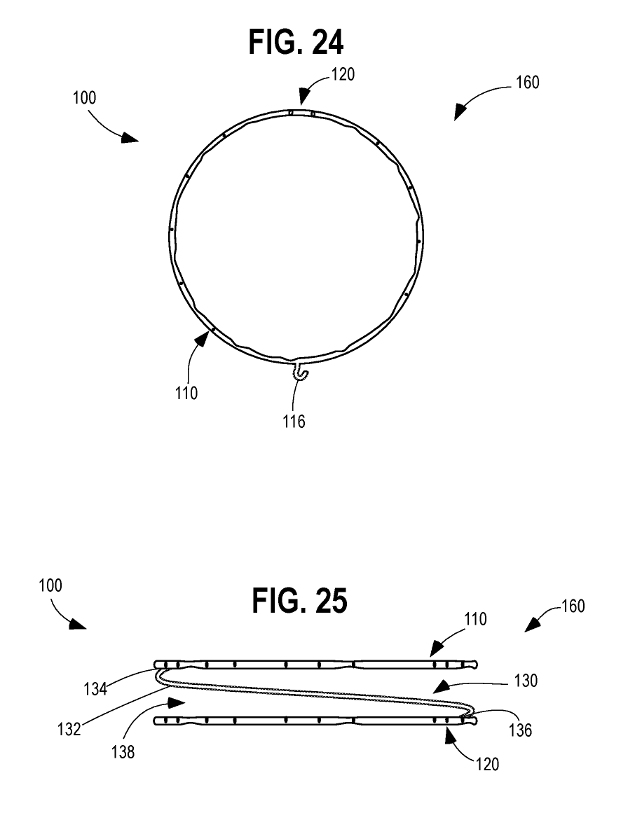

FIGS. 6-25 depict exemplary occlusion or closure systems. According to one embodiment of the present invention, the system 100 can comprise a unitary structure that generally comprises a plurality of frames, which can include, without limitation, a distal frame 110, a proximal frame 120, and a connection frame 130. In some embodiments, either or both of the distal frame 110 and the proximal frame 120 may be comprised of the apparatus 50, or some form thereof, where the system 100 would generally comprise at least one apparatus 50 attached to the connection frame 130. In a preferred embodiment, the plurality of frames can comprise integrally connected or continuously attached wire or tubular structure. The diameter or thickness of the wire or tubular structure may be generally proportional to the size of the system 100. In some embodiments, the plurality of frames can be formed of a flexible and manipulatable biocompatible or bioresorbable material or polymer, including, without limitation, iron, magnesium, platinum, stainless steel, cobalt-chromium-nickel alloy, or nickel titanium metal alloy, such as nitinol. In a preferred embodiment, the distal frame 110 and proximal frame 120 can comprise a plurality of notches or cut-outs 112, 122 along their lengths and at regular or irregular intervals. In one embodiment, the plurality of frames can be collapsible or capable of being distorted so that the system 100 can be, among other things, deployable via transcatheter techniques. In this way, nitinol is preferable because of its desirable characteristics that allow the system 100 to be compressed during the delivery process, while returning to its intended shape in vivo, as previously explained herein. It will be understood that the plurality of frames can comprise or be formed of any suitable material, polymer, or combination of materials and/or polymers that is, at the least, biocompatible and capable of adequate shape-memory. Further, in another embodiment, the system 100 may contain one or more sensors, including, but not limited to electrical sensors, pressure sensors, and biochemical sensors, impact sensors, strain gauges, accelerometers, non-physical contact sensors, acoustic sensors, infrared sensors, and other sensors.

As used herein, “distal” refers to the general direction of deployment, such as near a delivery device 170 deployment location, and “proximal” refers to the general direction away from deployment, such as away from a delivery device 170 deployment location.

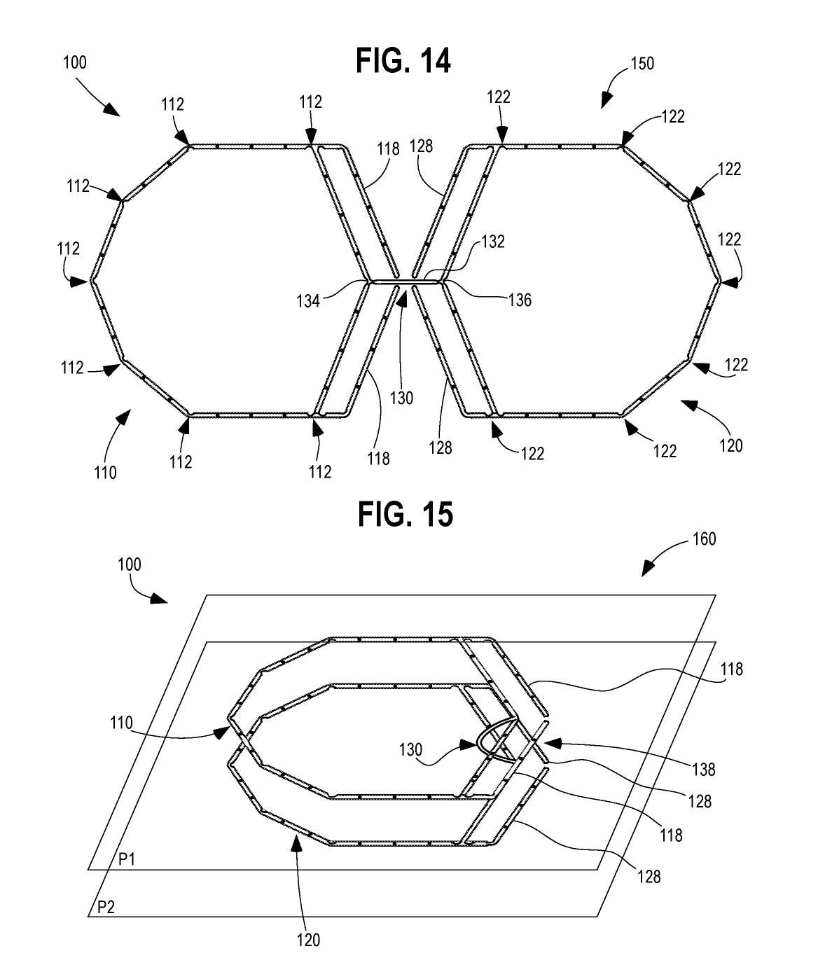

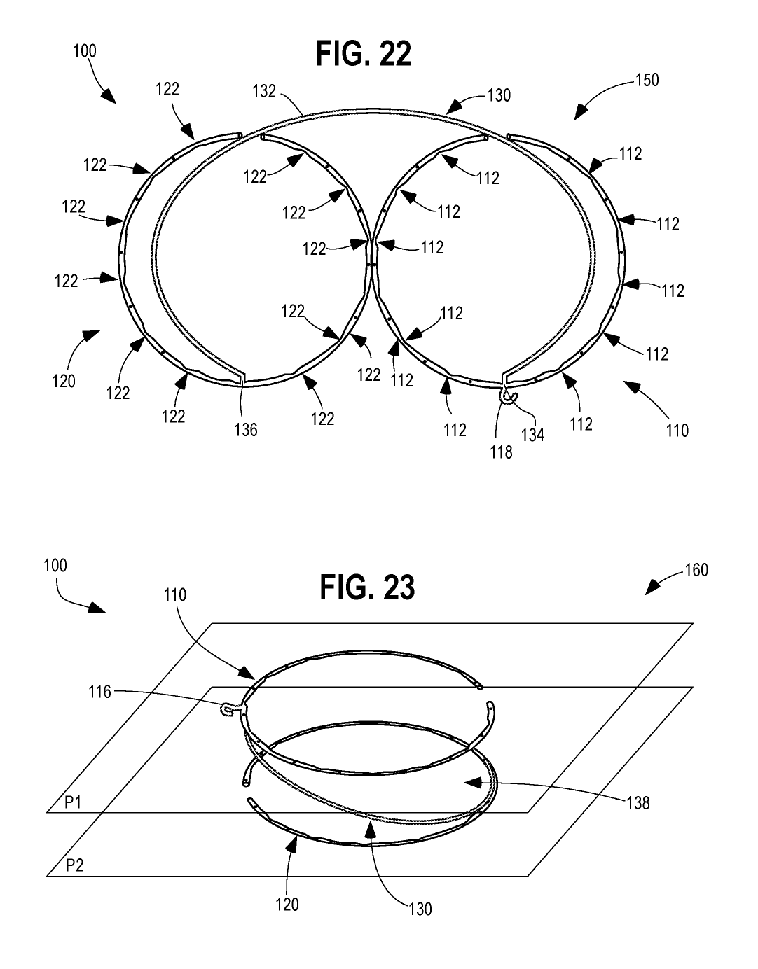

In certain embodiments, the distal frame 110 and proximal frame 120 can be a variety of symmetrical or asymmetrical geometric shapes or sizes suitable to achieve the purposes of the present invention. In some embodiments of the present invention, the distal frame 110 and the proximal frame 120 may generally define annular or substantially closed shapes, as best illustrated in FIGS. 6 and 22. In other embodiments, the distal frame 110 and the proximal frame 120 may generally define less-circular shapes, as best illustrated in FIG. 14. The distal frame 110 and proximal frame 120 may, in some embodiments, generally define a first plane P₁ and a second plane P₂, respectively. It will be understood, however, that in other embodiments, one or neither of the distal frame 110 or proximal frame 120 may generally define a plane. In some embodiments, the distal frame 110 and proximal frame 120 of the system 100 can assume the same or generally similar shape. In other embodiments, the distal frame 110 and proximal frame 120 of the system 100 can assume distinct or different shapes. In one embodiment, the flexibility of the plurality of frames allows for the system 100 to be expanded, contracted, and positionally adjusted to accommodate bodily passageways of varying shape and size. In some embodiments, the flexibility of the plurality of frames also allows for the distal frame 110 and the proximal frame 120 to be offset at an angle from the subject bodily passageway, thereby providing improved stability and continuity for the system 100. In some embodiments, the first plane P₁ and the second plane P₂ may be aligned in parallel or perpendicularly, or any other orientation. In another embodiment, configuring one of the frames at an angle relative to the bodily passageway advantageously allows for another frame to be properly deployed and implanted.

In a preferred embodiment of the present invention, the shape and size of the distal frame 110 and proximal frame 120 can correspond with the size of the subject bodily passageway, including a septal aperture 22, and may vary depending on the location of the bodily passageway in the body. For example, the shape and size of the distal frame 110 and proximal frame 120 may be designed to avoid any potential interaction or interference with surrounding bodily and/or medical structures, including, without limitation, the mitral valve, tricuspid valve, and pulmonary veins. In some embodiments, the distal frame 110 and proximal frame 120 of the system 100 can be generally the same size. In other embodiments, the distal frame 110 and proximal frame 120 can be different sizes. Preferably the distal frame 110 and proximal frame 120 each have a diameter generally ranging from 10 mm to 40 mm in size. However, it will be understood that the diameters of the distal frame 110 and proximal frame 120 can range from 50 mm to 4 mm.

In some embodiments, at least one of the plurality of frames, including, but not limited to, the distal frame 110, the proximal frame 120, and the connection frame 130, may further comprise at least one flexible biocompatible membrane or membranous covering 114, 124 attached to and covering at least a portion thereof that is adapted to adequately occlude or close the subject bodily passageway. In one embodiment, the distal frame 110 or the proximal frame 120 is attached to and desirably extends substantially around the periphery of the membrane or the membranous covering 114, 124 to hold the membrane or the membranous covering 114, 124 generally taut. The membrane or membranous covering 114, 124 may be attached to plurality of frames by any suitable attachment means presently known or later discovered, including, without limitation, via sutures, an adhesive, gluing, welding, or the like. For example, in one embodiment, the distal frame 110 or proximal frame 120 may comprise holes spaced apart, preferably approximately 2 mm to 3 mm apart, that allow for the membrane or membranous covering 114, 124 to be attached to distal frame 110 or proximal frame 120.

Similar to the membrane or membranous covering 56, the membrane or membranous covering 114, 124 preferably comprise pericardium, PTFE, or ePTFE for the same or similar advantageous material characteristics. In a preferred embodiment, the membrane or membranous covering 114, 124 is adapted to inhibit the passage of blood while also permitting post-deployment transseptal punctures and interatrial re-entry. According to some embodiments, the membrane or membranous covering 114, 124 can comprise any natural, synthetic, or bioengineered material, whether presently known or later discovered, that resiliently occludes the passageway and is also sufficiently durable to allow for post-deployment medical punctures without jeopardizing the integrity of the membrane or the membranous covering 114, 124, the system 100, or the system's 100 ability to occlude.

The plurality of notches or cut-outs 112, 122 of the distal frame 110 and proximal frame 120 permit for certain material reaction and behaviors that generally reduce the strain and related fatigue on the distal frame 110, proximal frame 120, the system 100, and the membrane or membranous coverings 114, 124 resulting from the cyclic loading of forces on the same. This resulting reduction of strain and fatigue on the distal frame 110, proximal frame 120, the system 100, and the membrane or membranous coverings 114, 124 greatly reduces the propensity of the system 100 to tear or fracture, which is an improvement over known occlusion or closure devices. Further, the less-circular shape of the distal frame 110 and the proximal frame 120, as best illustrated in FIG. 14, may further reduce the strain and fatigue on the system 100 and the membrane or membranous coverings 114, 124, because the less-circular shapes of the distal frame 110 and the proximal frame 120 do not require the membrane or membranous coverings 114, 124 to overly elongate when the system 100 is moved between the first position 150, the second position 160, and any other position.

The plurality of notches or cut-outs 112, 122 can further facilitate the additional flexibility of the system 100. Specifically, the plurality of notches or cut-outs 112, 122 allow the distal frame 110 and proximal frame 120 to selectively deform more easily, which is advantageous for purposes of overcoming the limited flexibility of the membrane or membranous coverings 114, 124 or other elements of the system 100. Such flexibility can allow the system 100 to adequately and repeatedly move between various positions without requiring of the membrane or membranous coverings 114, 124 to be slack or comprise excess or additional material for purposes of overcoming its limited flexibility.

In some embodiments, the distal frame 110 or the proximal frame 120 may comprise at least one attachment system 116, as best illustrated in FIGS. 6-8, 10, 11, and 22-24. The attachment system 116 adapted for attaching to a wire or cable for pulling the system 100 into a delivery device 170, such as a catheter or sheath, prior to delivery or deployment of the system 100. Although the attachment system 116 is depicted as a hook, it will be understood that the attachment system 116 may comprising any attachment means designed to achieve the purposes of the present invention, whether presently known or later developed, including a straight or curved protrusion. Further, it will be understood that although the attachment system 116 is depicted as attached to the distal-most portion of the system 100, it will be understood that the attachment system 116 may be attached at any other suitable location on the system 100, including at the proximal-most portion of the system 100, any lateral portion of the system 100, or at any position along the lengths of the frames of the system 100.

The connection frame 130 can comprise an at least one connection element 132, including a wire or tubular structure, that connects or affixes to the distal frame 110 at an at least one location 134 along the length or circumference thereof, whether symmetrical or asymmetrically. The at least one connection element 132 may further connect or affix to the proximal frame 120 at an at least one corresponding or non-corresponding location 136 along the length or circumference thereof, whether symmetrical or asymmetrically. Any suitable number of connection elements 132 may be employed in a preferred embodiment of the present invention. The connection frame 130 can assume several shapes or arrangement, as best illustrated in FIGS. 6, 7, 12, 14, 15, 22, and 23.

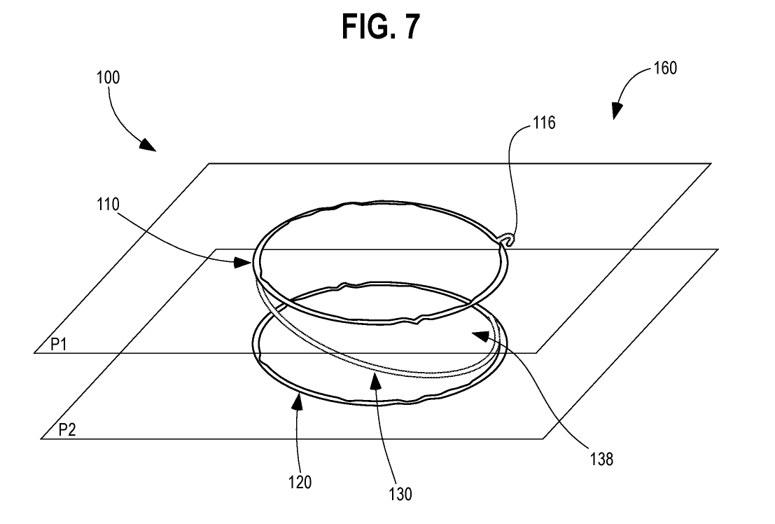

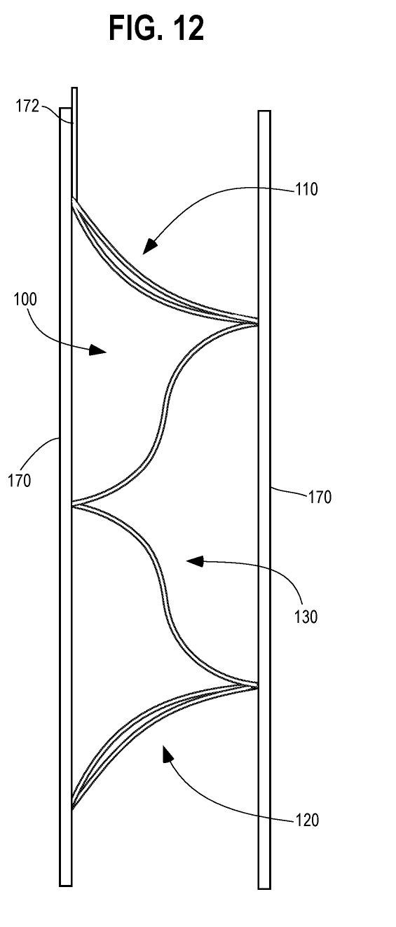

In one embodiment of the present invention, the system 100 is adapted to elastically move or distort between a series of shapes, including a first position 150 and a second position 160. The first position 150 can be alignable in a substantially elongated shape, as best illustrated in FIGS. 6,14, and 22. The elongated first position 150 may be suitable for loading in a delivery device 170, such as a catheter or sheath. As best illustrated in FIG. 12, the system 100 can be collapsible and percutaneously deployable via a delivery device 170, such as a catheter or sheath, generally in the first position 150. In the first position 150, the distal frame 110 and the proximal frame 120 can be maintained in a specified spaced relationship, including, without limitation, a radially spaced relationship and/or a spaced relationship in which the distal frame 110 and the proximal frame 120 are concentrically aligned. The second position 160 can generally be the intended shape for deployment and engagement of the system 100, such as a generally longitudinally compressed shape, where the distal frame 110 and the proximal frame 120 can be maintained in a specified spaced relationship, including, without limitation, an axially spaced and/or concentrically aligned relationship, as best illustrated in FIGS. 7-9, 15-18, and 23-25. In one embodiment of the present invention, the system 100 can be adapted so that it is biased to assume and/or maintain the intended shape, such as the shape of the second position 160, especially when the system 100 is free of any restraining forces caused by a delivery device 170 or the like. The biased position of the system 100 being achievable through a variety of means, including, without limitation, using a material with shape-memory capabilities resulting from a heat-setting or similar annealing process.

Although the delivery system 170 is described and depicted as a catheter or sheath, it will be understood that the delivery system 170 can comprise any suitable delivery means, presently known or later discovered, such as a delivery hub, sheath, or the like, and the system 100 can be attached or deployed by any other delivery means, whether presently known or later discovered, including, without limitation, being attached to a delivery hub. Further, as best illustrated in FIG. 12, the delivery system 170 may comprise a delivery cable 172 coupled with or attached to the system 100 to facilitate the release or deployment of the same. It will be understood that although the delivery cable 172 is depicted as attached to the distal-most portion of the system 100, the delivery cable 172 may be attached at any other suitable location on the system 100, including at the proximal-most portion of the system 100, any lateral portion of the system 100, or at any position along the lengths of the frames of the system 100. Further yet, it will be understood that the delivery device 170 of the present invention can be adapted for use with adult patients and child patients.

The spaced relationships of the distal frame 110 and the proximal frame 120 in the first position 150 and the second position 160 can be achieved via the connection frame 130 and the various shapes and arrangements thereof. To achieve the first position 150 of the system 100, the connection frame 130 can be adapted to achieve a generally linear shape or a relatively curved shape, among any other suitable shape, as best illustrated in FIGS. 6, 12, 14, and 22. To achieve the second position 160 of the system 100, the connection frame 130 can be adapted to assume several shapes. In some embodiments, the connection frame 130 can assume a generally spiraled or coiled shape in the second position 160, as best illustrated in FIGS. 7, 10, 23. In other embodiments, the connection frame 130 can assume an arch-like or generally “C” shape in the second position 160, as best illustrated in FIGS. 15-18. However, it will be understood that the connection frame 130 can be adapted to achieve any number of shapes in the first position 150 or the second position 160. The connection frame 130 can be heat-set to achieve the desired shape and material behavior to maintain the spaced relationship of the distal frame 110 and the proximal frame 120 in either the first position 150 or the second position 160. However, it will be understood that any method or means can be used to achieve the desired shape and material behavior of the connection frame 130.

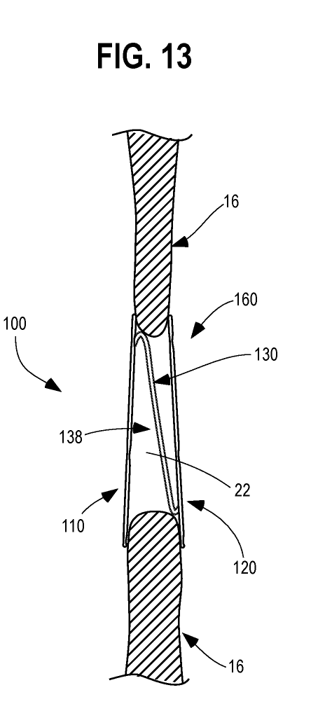

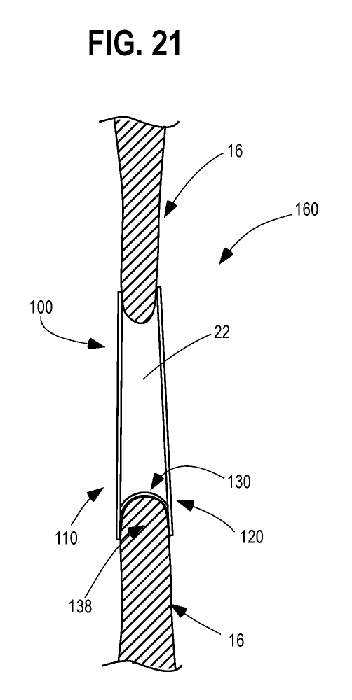

The anchor method or means for attaching the system 100 adjacent to the subject bodily passageway can take several different forms and may be adapted to permit the system 100 to be self-centering about the subject bodily passageway. In one embodiment, the plurality of frames of the system 100 may be urged at least partially, and entirely in some cases, against the edges of the septal wall defining the subject bodily passageway to inhibit the flow of liquids, including blood, therethrough. The forces exerted by the plurality of frames of the system 100 can arise from the well-defined shape-memory, pronounced elasticity, and resilient nature of the material comprising the system 100. In a preferred embodiment, in the second position 160, the connection frame 130 can serve as an attachment means for attaching to the subject bodily passageway and sealingly engaging the distal frame 110 and proximal frame 120 with the septal wall for purposes of inhibiting the flow of liquids, including blood, through the bodily passageway. In most embodiments, the connection frame 130 defines a securing region 138 in the second position 160, which can be disposed intermediate to the septal wall. Further, the connection frame 130 can be shaped from and comprise a material with adequate flexibility, resilience, and shape-memory to sealingly engage and effectively clasp, via the securing region 138, the septal wall of the heart 10 or any appendage thereof for purposes adequately anchoring the system 100. As best illustrated in FIG. 13, the spiraled or coiled shape of the connection frame 130 defining the securing region 138 has a self-expanding nature and sealing engages the septum 16 of the heart 10 in a threaded or screw-like manner. As best illustrated in FIG. 21, the arch-like or generally “C” shape of the connection frame 130 defining the securing region 138 sealing engages the septum 16 of the heart 10 in a clasping or clamping-like manner. However, it will be understood that the non-parallel alignment of the first plane P₁ and the second plane P₂, as best illustrated in FIG. 18, can result in compressive forces created by the arch-like or generally “C” shape of the connection frame 130. These compressive forces, especially at the portions of the distal frame 110 and proximal frame 120 most opposite of the connection frame 130, can cause the distal frame 110 and the proximal frame 120 to be urged against the septal wall defining the subject bodily passageway to inhibit the flow of liquids therethrough and also effectively serve as an attachment means or a complementary attachment means.

As best illustrated in FIGS. 7, 8, 23, and 24, the spiraled or coiled shape of the connection frame 130 in the second position 160, in some embodiments, may comprise an arc that is generally concentric with the shape of the distal frame 110 and/or the proximal frame 120. Further, the arc can have a length that is proportional to the circumference of the annular shape of the distal frame 110 and/or the proximal frame 120. For example, FIG. 7 depicts the arc of the connection frame 130 relative to the distal frame 110 and/or the proximal frame 120 as approximately fifty percent (50%) of the circumference of the annular shape of either the distal frame 110 or the proximal frame 120. However, it will be understood, that the arc of the connection frame 130 can assume any proportion of the circumference of the annular shape of the distal frame 110 and/or the proximal frame 120. In one embodiment, the arc of the connection frame 130 can be between about five percent (5%) and four hundred percent (400%) of the circumference of the annular shape of the distal frame 110 and/or the proximal frame 120. In another embodiment, the arc of the connection frame 130 can be between about twenty-five percent (25%) and three hundred percent (300%) of the circumference of the annular shape of the distal frame 110 and/or the proximal frame 120. In a further embodiment, the arc of the connection frame 130 can be between about fifty percent (50%) and one hundred percent (100%) of the circumference of the annular shape of the distal frame 110 and/or the proximal frame 120. In a further embodiment, the arc of the connection frame 130 can be about seventy-five percent (75%) of the circumference of the annular shape of the distal frame 110 and/or the proximal frame 120.

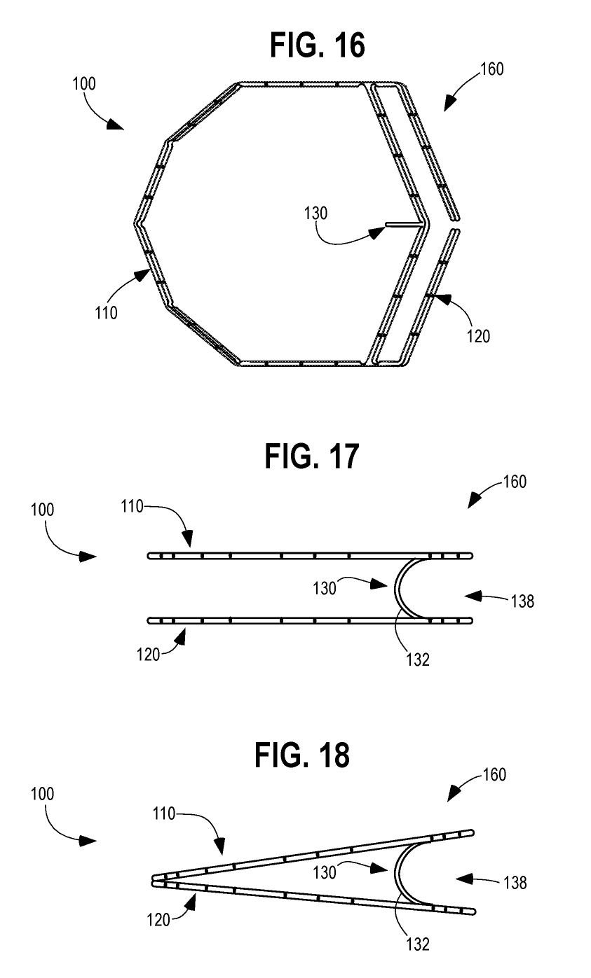

As best illustrated in FIGS. 15-18, the arch-like or generally “C” shape of the connection frame 130 in the second position 160, in other embodiments, may define a profile that is relatively small relative to the profile of the distal frame 110 and/or the proximal frame 120. For example, the profile of the connection frame 130 in the second position 160, as illustrated in FIG. 16, extends minimally into the center of the profile of the distal frame 110 and/or the proximal frame 120. Although the orientation of the connection frame 130 is depicted as extended directly inward of the system 100 in FIGS. 15-18, it will be understood that the connection frame 130 may assume any orientation relative to the system 100, the distal frame 110, and/or the proximal frame 120 and may be any size or shape necessary to achieve the purposes of the present invention.

Further, as best illustrated in FIGS. 15-18, in some embodiments, the distal frame 110 and the proximal frame 120 may further comprise a plurality of expansion legs 118, 128 that aid in holding the membrane or the membranous covering 114, 124 generally taut. In a preferred embodiment, the plurality of expansion legs 118, 128 extend beyond the horizontal profile of the securing region 138 defined by the connection frame 130, as best illustrated in FIGS. 15-18. The extension of the plurality of the extension legs 118, 128 beyond the horizontal profile of the securing region 138 aids in the ability of the system 100 to self-center about the subject bodily passageway, ensures an adequate fit with the bodily passageway, and sealingly engages the bodily anatomy of the patient. Further, the size of the region defined by the broad profiles of the distal frame 110 and the proximal frame 120 expand with the addition of the plurality of the extensions legs 118, 128, such that the size of the covering profile of the membrane or the membranous covering 114, 124 permits the system 100 to adequately occlude or close larger bodily passageways than what is currently permitted by other known occlusion or closure devices or techniques of similar share and size.

The present invention also contemplates a method for making the apparatus 50 and the system 100. According to one embodiment of the present invention, the method for making apparatus 50 or the system 100 described herein can generally comprise the steps of providing a suitable material, cutting the desired shape of the apparatus 50 or the system 100 from the material, and heat-setting all or portion of the cut material to achieve the desired self-expanding shape-memory nature of the apparatus 50 or system 100. Although the step of cutting the desired shape of the apparatus 50 or the system 100 from the material can include die cutting or laser cutting, it will be understood that any suitable cutting means can be used, including those presently known or later developed.

The present invention also contemplates a method for delivering the apparatus 50 and the system 100 to occlude or close a bodily passageway. A preferred method, and non-limiting example, for deploying the apparatus 50 and system 100 is described for inhibiting blood flow through an aperture 22 in the heart 10. Generally, the method of delivery consists of a transcatheter treatment technique method, which eliminates the need for open-heart surgery, comprising the steps of placing the apparatus 50 or system 100 adjacent to the aperture 22 after extending the delivery device 170 through the aperture 22, such that the distal portion of the apparatus 50 or system 100 is located in an atrium of the heart, such as the left atrium 14, and the proximal portion of the same is located in an atrium of the heart, such as the right atrium 12.

According to another embodiment of the present invention, the method for occluding or closing a bodily passageway 200, such as an ASD, generally comprises releasing and anchoring or implanting the apparatus 50 or system 100 adjacent to a bodily passageway to inhibit or prevent the flow of blood therethrough. In a preferred embodiment, the system 100 may extend through an ASD such that the distal frame 110 is located in one atrium, such as the left atrium 14, and the proximal frame 120 is located in the other atrium, such as the right atrium 12. According to one embodiment, the apparatus 50 or system 100 may be delivered percutaneously via a delivery device, including the delivery device 170.

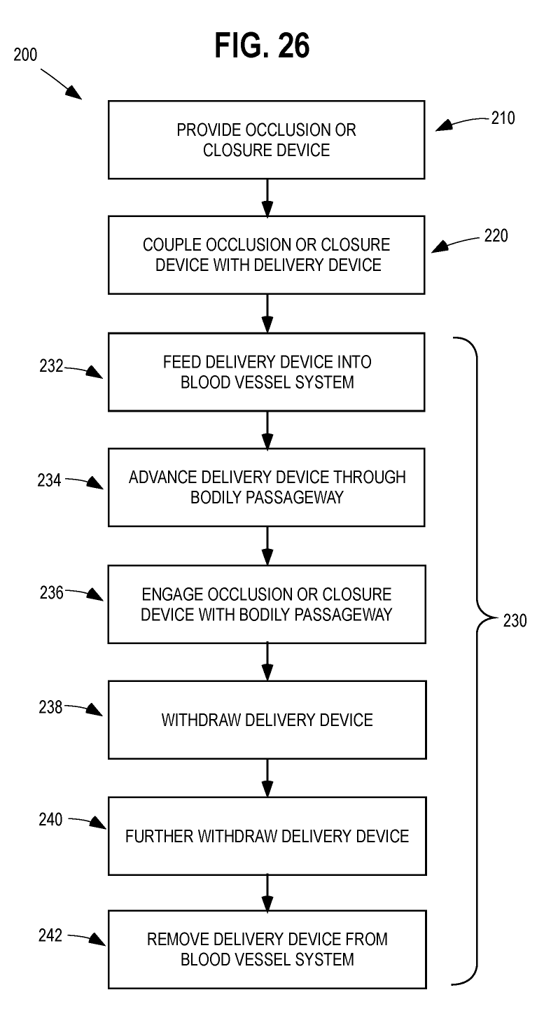

As illustrated in FIG. 26, the method 200 may comprise the specific steps of providing an occlusion or closure device 210, including the apparatus 50 or system 100, coupling the occlusion or closure device with a delivery device 220, and deploying the occlusion or closure device 230 at or adjacent to the desired point in the patient's body. Where, the step of deploying the occlusion or closure device 230 further comprises the steps of feeding the delivery device into the blood vessel system 232, advancing the delivery device through the subject bodily passageway 234, engaging the occlusion or closure device with the subject bodily passageway 236, withdrawing the delivery device 238, further withdrawing the delivery device 240, and removing the delivery device from the blood vessel system 242.

Specifically, the step of advancing the delivery device through the subject bodily passageway 234 can comprise the step of advancing the delivery device along a guide wire that has been inserted from a venous access point and directed across the bodily passageway. Once a guide wire is advanced to an intended position near or adjacent to the bodily passageway, the delivery device can be inserted over the guide wire across the bodily passageway. The step of engaging the occlusion or closure device with the subject bodily passageway 236 can comprise removing the guide wire and inserting the occlusion or closure device in its elongated position, as shown in FIG. 12, through the delivery device and across the bodily passageway. The step of engaging the occlusion or closure device with the subject bodily passageway 236 can further comprise the step of rotating the occlusion or closure device as it is released from the delivery device to ensure appropriate apposition to the left side of the bodily passageway, including the inter-atrial septum 16, and to avoid interactions with surrounding structures. Once the occlusion or closure device is in the correct orientation and position, the step of engaging the occlusion or closure device with the subject bodily passageway 236 can comprise the step of tractioning the occlusion or closure device to be apposed to the bodily passageway. It will be understood, however, that the occlusion or closure device may be fully released from the delivery device during the steps of withdrawing the delivery device 238 and/or further withdrawing the delivery device 240. In this way, once the distal portion of the occlusion or closure device is tractioned to be apposed to the bodily passageway, withdrawing the delivery device 238 and/or further withdrawing the delivery device 240 can extract the other portions of the occlusion or closure device, including the proximal portion thereof, to sealingly engage the bodily passageway. Once the occlusion or closure device sealingly engages the bodily passageway, the step of removing the delivery device from the blood vessel system 242 can be completed.

Regarding the method for occluding or closing a bodily passageway 200, the delivery device 170 can be fed percutaneously through the femoral vein, the inferior vena cava, or through a vein in the upper torso, such as the jugular vein, the superior vena cava. In a preferred embodiment, the system 100 may collapse as it is inserted into the delivery device 170, which may cause the system 100 to generally assume the first position 150, as best illustrated depicted in FIG. 12. In some embodiments, deploying the apparatus 50 or system 100 may comprise the steps of advancing the apparatus 50 or system 100 through the delivery device 170 by using a percutaneously adaptable actuator, such that the apparatus 50 or system 100 is free to assume its intended, biased shape. In another embodiment, deploying the apparatus 50 or system 100 may comprise the step of pulling the delivery device 170 away from the delivery site while maintaining the position of the apparatus 50 or system 100.

In a preferred embodiment, when deploying the system 100, the delivery device 170 traverses the subject bodily passageway, and the system 100 will be partially advanced from the delivery device 170 so that the distal frame 110, including any covering attached thereto, begins to assume its intended shape via the self-expanding shape-memory nature of the material comprising the distal frame 110. In this manner, the system 100, only partially assumes the second position 160 until the system 100 is fully advanced from the delivery device 170, which is preferably achieved once the delivery device 170 has fully traversed the subject bodily passageway. When the proximal frame 120 is advanced out of the delivery device 170, the delivery device 170 can be removed from the patient's body, and the system 100 can remain in the body at the location of the bodily passageway. Once deployed, the securing region 138 defined by the connection frame 130 anchors the system 100 in place against the septal wall, as described herein, including via threaded engagement or in a clasping manner, and in a self-centering manner. Full deployment of the system 100 may substantially or completely occlude the subject bodily passageway.

In one embodiment of the present invention, the apparatus 50 or system 100 may be retrieved or recaptured from the delivery site by a variety of means. In one embodiment, retrieving or recapturing the apparatus 50 or system 100 may comprise the step of pulling the apparatus 50 or system 100 into a retrieving device, which may be similar to the delivery device 170 described herein. In another embodiment, retrieving or recapturing the apparatus 50 or system 100 may comprise the step of advancing the retrieving device toward from the retrieving site, which may be the former delivery site, while maintaining the position of the apparatus 50 or system 100.

It will be understood that certain steps of the method for occluding or closing a bodily passageway 200 may vary in certain embodiments. It will also be understood that certain steps of the method 200 may occur in a different order that represented or depicted herein. Further, it will similarly be understood that certain steps of the method 200 may occur simultaneously with one another.

From the foregoing, it will be seen that the various embodiments of the present invention are well adapted to attain all the objectives and advantages hereinabove set forth together with still other advantages which are obvious and which are inherent to the present structures. It will be understood that certain features and sub-combinations of the present embodiments are of utility and may be employed without reference to other features and sub-combinations. Since many possible embodiments of the present invention may be made without departing from the spirit and scope of the present invention, it is also to be understood that all disclosures herein set forth or illustrated in the accompanying drawings are to be interpreted as illustrative only and not limiting. The various constructions described above and illustrated in the drawings are presented by way of example only and are not intended to limit the concepts, principles and scope of the present invention.

Many changes, modifications, variations and other uses and applications of the present invention will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. An apparatus for occluding a bodily passageway, the apparatus comprising:
a structure comprising a shape-memory metal alloy; and
a membranous material attached to the structure;
wherein the structure is adapted to be deployed adjacent to the bodily passageway;
wherein the structure is adapted to sealingly engage the bodily passageway.

2. The apparatus of claim 1, wherein the structure further comprises a plurality of notches.

3. The apparatus of claim 1 further comprising an at least one sensor attached to the apparatus.

4. A system for occluding a bodily passageway, the system comprising:
a structure comprising a shape-memory metal alloy movable between a first position and a second position comprising:
a plurality of frames; and
an at least one membranous material attached to the plurality of frames;
wherein the plurality of frames define a distal end and a proximal end;
wherein the distal end and the proximal end are adapted to be deployed adjacent to the bodily passageway;
wherein the structure is adapted to sealingly engage the bodily passageway.

5. The system of claim 4, wherein the plurality of frames comprises a distal frame, a proximal frame, and a connection frame.

6. The system of claim 5, wherein the distal frame and the proximal frame further comprise a plurality of expansion legs.

7. The system of claim 5, wherein the connection frame is a generally linear shape in the first position.

8. The system of claim 5, wherein the connection frame defines a securing region in the second position.

9. The system of claim 8, wherein the connection frame is a spiraled shape in the second position.

10. The system of claim 8, wherein the connection frame is an arch-like shape in the second position.

11. The system of claim 4, wherein the plurality of frames further comprise a plurality of notches.

12. The system of claim 4 further comprising an at least one sensor attached to the system.

13. A method for making an occlusion device, the method comprising:
providing a shape-memory metal alloy;
cutting a desired shape of the occlusion device from the shape-memory metal alloy;
heat-setting the desired shape of the occlusion device; and
attaching a membranous material to the occlusion device.

14. The method of claim 13, wherein the occlusion device comprises a plurality of notches.

15. The method of claim 13, wherein the occlusion device comprises an at least one sensor attached to the occlusion device.

16. A method for occluding a bodily passageway, the method comprising:
providing an occlusion device, the occlusion device comprising a structure comprising a shape-memory metal alloy and a membranous material attached to the structure, wherein the structure is adapted to sealingly engage the bodily passageway;
coupling the occlusion device with a delivery device; and
deploying the occlusion device.

17. The method of claim 16, wherein the delivery device comprises a delivery catheter.

18. The method of claim 16, wherein the step of deploying the occlusion device further comprises the steps of:
feeding the delivery device into a blood vessel system;
advancing the delivery device through the bodily passageway;
engaging the occlusion device with the bodily passageway;
withdrawing the delivery device; and
removing the delivery device from the blood vessel system.

19. The method of claim 18, wherein the step of deploying the occlusion device further comprises the step of withdrawing the delivery device further.

20. The method of claim 18, wherein the step of engaging the occlusion device with the bodily passageway further comprises the step of anchoring the occlusion device by a securing region defined by the occlusion device.

21. The method of claim 20, wherein the securing region defined by the occlusion device sealing engages the bodily passageway.

22. The method of claim 18, wherein the step of engaging the occlusion device with the bodily passageway further comprises the step of rotating the occlusion device as it exits the delivery device to ensure appropriate apposition relative to the bodily passageway.

23. The method of claim 16 further comprising the step of retrieving the occlusion device.

24. The method of claim 16, wherein the occlusion device comprises a plurality of notches.

25. The method of claim 16, wherein the occlusion device comprises an at least one sensor attached to the occlusion device.
